(19) United States
(12) Patent Application Publication
(10) Pub. No.: US 2022/0064635 A1
(43) Pub. Date: Mar. 3, 2022

(54) CRISPR COMPOSITIONS AND METHODS FOR PROMOTING GENE EDITING OF ADENOSINE DEAMINASE 2 (ADA2)

(71) Applicant: EmendoBio Inc., Wilmington, DE (US)

(72) Inventors: David Baram, Tel Aviv (IL); Asael Herman, Ness-Ziona (IL); Rafi Emmanuel, Ramla (IL); Michal Golan Mashiach, Ness-Ziona (IL); Joseph Georgeson, Rehovot (IL)

(73) Assignee: EmendoBio Inc., Wilmington, DE (US)

(21) Appl. No.: 17/420,934

(22) PCT Filed: Jan. 6, 2020
(86) PCT No.: PCT/US2020/012402

§ 371 Date: Jul. 6, 2021

Related U.S. Application Data
(60) Provisional application No. 62/789,275, filed on Jan. 7, 2019.

(51) Int. Cl. C12N 15/11; C12N 9/22; C12N 15/90; A61K 35/28

(52) U.S. Cl. CPC C12N 15/11; C12N 9/22; C12N 2310/20; A61K 35/28; C12N 15/907

(57) ABSTRACT

RNA molecules comprising a guide sequence portion having 17-25 nucleotides in the sequence of 20-22 contiguous nucleotides set forth in any one of SEQ ID NOs: 1-12655 and compositions, methods, and uses thereof.

This application claims the benefit of U.S. Provisional Application No. 62/789,275, filed Jan. 7, 2019 the contents of which is hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “200106_90792-A-PCT_Sequence_Listing_DH.txt”, which is 2,303 kilobytes in size, and which was created on Jan. 6, 2020 in the IBM-PC machine format, having an operating system compatibility with MS-Windows, which is contained in the text file filed Jan. 6, 2020 as part of this application.

### BACKGROUND OF INVENTION

A genetic disorder is caused by one or more abnormalities in the genome. Genetic disorders may be regarded as either “dominant” or “recessive.” Recessive genetic disorders are those which require two copies (i.e., two alleles) of the abnormal/defective gene to be present. In contrast, a dominant genetic disorder involves a gene or genes which exhibit(s) dominance over a normal (functional/healthy) gene or genes. As such, in dominant genetic disorders only a single copy (i.e., allele) of an abnormal gene is required to cause or contribute to the symptoms of a particular genetic disorder. Such mutations include, for example, gain-of-function mutations in which the altered gene product possesses a new molecular function or a new pattern of gene expression. Other examples include dominant negative mutations, which have a gene product that acts antagonistically to the wild-type allele.

### Adenosine Deaminase 2 (ADA2) Deficiency

Adenosine deaminase 2 (ADA2) deficiency is caused by mutations in the ADA2 gene which severely reduce or eliminate the activity of adenosine deaminase 2. This condition is inherited in an autosomal recessive pattern. Adenosine deaminase 2 (ADA2) deficiency is characterized by abnormal inflammation of various tissues. Signs and symptoms can begin anytime from early childhood to adulthood. The severity of the disorder also varies, even among affected individuals in the same family.

### SUMMARY OF THE INVENTION

Disclosed is an approach for repairing at least one allele bearing a disease-associated mutation (“mutant allele”) by utilizing an RNA guided DNA nuclease to edit/correct/modify the nucleic acid sequence of the mutant allele such as to express a functional protein.

According to some embodiments, the present disclosure provides a method for treating, preventing or ameliorating a disease associated with a mutation in the ADA2 gene. In some embodiments, the disease-associated mutation is targeted. In some embodiments, the method further comprises the step of allele cleavage by a CRISPR nuclease. The allele cleavage is selected from the group consisting of: a double strand break (DSB) and a single strand break. In some embodiments, the CRISPR nuclease affects a double strand break (DSB). In some embodiments, the method further comprises the step of correction of the allele such that the corrected allele result in an expression of a functional ADA2 protein. In some embodiments, the correction is performed by homology directed repair (HDR).

According to embodiments of the present invention, there is provided an RNA molecule comprising a guide sequence portion having 17-25 nucleotides comprising the sequence of 20-22 contiguous nucleotides set forth in any one of SEQ ID NOs: 1-12655.

According to some embodiments of the present invention, there is provided a composition comprising an RNA molecule comprising a guide sequence portion having 17-25 nucleotides comprising the sequence of 20-25 contiguous nucleotides set forth in any one of SEQ ID NOs: 1-12655 and a CRISPR nuclease. In some embodiments, the composition further comprises a nucleic acid template for homology-directed repair, alteration, or replacement of a target sequence of an allele comprising the disease-associated mutation.

According to some embodiments of the present invention, there is provided a method for repairing/correcting a mutant ADA2 allele in a cell, the method comprising delivering to the cell a composition comprising an RNA molecule comprising a guide sequence portion having 17-25 nucleotides comprising the sequence of 20-22 contiguous nucleotides set forth in any one of SEQ ID NOs: 1-12655 and a CRISPR nuclease. In some embodiments, a nucleic acid template is further provided to the cell for homology-directed repair, alteration, or replacement of a target sequence of the mutant ADA2 allele.

According to some embodiments of the present invention, there is provided a method for treating, preventing or ameliorating ADA2 deficiency (DADA2) in a subject having DADA2, the method comprising delivering to the subject a composition comprising an RNA molecule comprising a guide sequence portion having 17-25 nucleotides comprising the sequence of 20-22 contiguous nucleotides set forth in any one of SEQ ID NOs: 1-12655 and a CRISPR nuclease. In some embodiments, a nucleic acid template is further provided to the cell for homology-directed repair, alteration, or replacement of a target DNA sequence comprising the pathogenic mutation.

In some embodiments, the method is performed ex vivo and the cell is provided/explanted from an individual patient. In some embodiments, the method further comprises the step of introducing the resulting cell, with the corrected/repaired/modified mutant ADA2 allele, into the individual patient (e.g. autologous transplantation).

According to some embodiments of the present invention, there is provided use of a composition comprising an RNA molecule comprising a guide sequence portion having 17-25 nucleotides comprising the sequence of 20-22 contiguous nucleotides set forth in any one of SEQ NOs: 1-12655 and a CRISPR nuclease for repairing/correcting/editing a mutant ADA2 allele in a cell, comprising delivering to the cell the composition comprising an RNA molecule comprising a guide sequence portion having 17-25 nucleotides comprising the sequence of 20-22 contiguous nucleotides set forth in any one of SEQ ID NOs: 1-12655 and a CRISPR nuclease.

According to embodiments of the present invention, there is provided a medicament comprising an RNA molecule comprising a guide sequence portion having 17-25 nucleotides comprising the sequence of 20-22 contiguous nucleotides set forth in any one of SEQ ID NOs: 1-12655 and a CRISPR nuclease for use in inactivating repairing/correcting/editing a mutant ADA2 allele in a cell, wherein the medicament is administered by delivering to the cell the composition comprising an RNA molecule comprising a guide sequence portion having 17-25 nucleotides comprising the sequence of 20-22 contiguous nucleotides set forth in any one of SEQ ID NOs: 1 -12655 and a CRISPR nuclease. In some embodiments, the medicament further comprises a nucleic acid template.

According to some embodiments of the present invention, there is provided use of a composition comprising an RNA molecule comprising a guide sequence portion having 17-25 nucleotides comprising the sequence of 20-22 contiguous nucleotides set forth in any one of SEQ ID NOs: 1-12655 and a CRISPR nuclease for treating ameliorating or preventing DADA2, comprising delivering to cells of a subject having or at risk of having DADA2, the composition comprising an RNA molecule comprising a guide sequence portion having 17-25 nucleotides comprising the sequence of 20-22 contiguous nucleotides set forth in any one of SEQ ID NOs: 1-12655 and a CRISPR nuclease.

According to some embodiments of the present invention, there is provided a medicament comprising the composition comprising an RNA molecule comprising a guide sequence portion having 17-25 nucleotides comprising the sequence of 20-22 contiguous nucleotides set forth in any one of SEQ ID NOs: 1-12655 and a CRISPR nuclease for use in treating ameliorating or preventing DADA2, wherein the medicament is administered by delivering to a subject having or at risk of having DADA2 the composition comprising an RNA molecule comprising a guide sequence portion having 17-25 nucleotides comprising the sequence of 20-22 contiguous nucleotides set forth in any one of SEQ NOs: 1-12655 and a CRISPR nuclease.

According to some embodiments of the present invention, there is provided a kit for correcting/repairing a mutant ADA2 allele in a cell, comprising an RNA molecule comprising a guide sequence portion having 17-25 nucleotides comprising the sequence of 20-22 contiguous nucleotides set forth in any one of SEQ ID NOs: 1-12655, a CRISPR nuclease, and optionally a tracrRNA molecule; and instructions for delivering the RNA molecule; CRISPR nuclease, and/or the tracrRNA to the cell. In some embodiment, the delivery is performed ex-vivo. In some embodiments, the delivery is performed within a subject's body. In some embodiments, the cells are HSC cell originated from the subject.

According to some embodiments of the present invention, there is provided a kit for treating DADA2 in a subject, comprising an RNA molecule comprising a guide sequence portion having 17-25 nucleotides comprising the sequence of 20-22 contiguous nucleotides set forth in any one of SEQ ID NOs: 1-12655, a CRISPR nuclease, and optionally a tracrRNA molecule; and instructions for delivering the RNA molecule; CRISPR nuclease, and optionally the tracrRNA to a subject having or at risk of having DADA2. According to some embodiments of the present invention, there is provided a kit for treating DADA2 in a subject, comprising an RNA molecule comprising a guide sequence portion having 17-25 nucleotides comprising the sequence of 20-22 contiguous nucleotides set forth in any one of SEQ ID NOs: 1-12655, a CRISPR nuclease, and optionally a tracrRNA molecule; and instructions for delivering the RNA molecule; CRISPR nuclease, and optionally the tracrRNA to cells of the subject having or at risk of having DADA2. In some embodiments, the cells are HSC cells obtained from the subject and the delivery is ex-vivo.

### BRIEF DESCRIPTION OF THE DRAWINGS

FIG. 1: Screen for activity of guides targeting ADA2 in Hela cells. spCas9 coding plasmid was co-transfected with each of the guide plasmids. Cells were harvested 72 h post DNA transfection. Genomic DNA was extracted and used for capillary electrophoreses using on-target primers which amplify the endogenous genomic regions. The graph represents the average of % editing±STDV of 3 independent experiments.

FIG. 2A: In vitro cleavage assay of guide 16, run on 1.7 agarose gel after proteinase K treatment, showing the full length template at 1000 bp (control) and the cleaved at 750 bp. * indicates non specific band.

FIG. 2B: In vitro cleavage assay of guide 12, run on 1.7% agarose gel after proteinase K treatment, showing the full length template at 1000 bp (control) and the cleaved at 500 bp.

### DETAILED DESCRIPTION

### Definitions

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The “guide sequence portion” of an RNA molecule refers to a nucleotide sequence that is capable of hybridizing to a specific target DNA sequence, e.g., the guide sequence portion has a nucleotide sequence which is fully complementary to the target DNA sequence. In some embodiments, the guide sequence portion is 17, 18, 19, 20, 21, 22, 23, 24 or 25 nucleotides in length, or approximately 17-25, 17-24, 18-22, 19-22, 18-20, 17-20, 21-22, 20-23, or 17-22 nucleotides in length. The guide sequence portion may be part of an RNA molecule that can form a complex with a CRISPR nuclease with the guide sequence portion serving as the DNA targeting portion of the CRISPR complex. When the DNA molecule having the guide sequence portion is present contemporaneously with the CRISPR molecule the RNA molecule is capable of targeting the CRISPR nuclease to the specific target DNA sequence. Each possibility represents a separate embodiment. An RNA molecule can be custom designed to target any desired sequence.

In embodiments of the present invention, an RNA molecule comprises a guide sequence portion having 17-25 nucleotides comprising the sequence of 20-22 contiguous nucleotides set forth in any one of SEQ ID NOs: 1-12655. In embodiments of the present invention, an RNA molecule comprises a guide sequence portion having 17-22 nucleotides in the sequence of 20-22 contiguous nucleotides set forth in any one of SEQ ID NOs: 1-12655.

As used herein, “contiguous nucleotides” set forth in a SEQ ID NO refers to nucleotides in a sequence of nucleotides in the order set forth in the SEQ ID NO without any intervening nucleotides. Sequences comprising the SEQ ID NO in which one or more of the nucleotides is chemically modified (i.e., having a backbone modification) are also encompassed.

Exemplary modifications to polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. As described herein, an example of a modified polynucleotide is an mRNA containing 1-methyl pseudo-uridine. For examples of modified polynucleotides and their uses, see U.S. Pat. No. 8,278,036. PCT International Publication No. WO/2015/006747, and Weissman and Kariko, 2015, (9):1416-7, hereby incorporated by reference.

In embodiments of the present invention, the guide sequence portion may be 20 nucleotides in length and consists of 20 nucleotides in the sequence of 20 contiguous nucleotides set forth in any one of SEQ ID NOs listed in Column 2 of Table 1. In embodiments of the present invention, the guide sequence portion may be 21 nucleotides in length and consists of 21 nucleotides in the sequence of 21 contiguous nucleotides set forth in any one of SEQ ID NOs listed in Column 3 of Table 1. In embodiments of the present invention, the guide sequence portion may be 22 nucleotides in length and consists of 22 nucleotides in the sequence of 22 contiguous nucleotides set forth in any one of SEQ ID NOs listed in Column 4 of Table 1.

In embodiments of the present invention, the guide sequence portion may be less than 20 nucleotides in length. For example, in embodiments of the present invention the guide sequence portion may be 17, 18, or 19 nucleotides in length. In such embodiments the guide sequence portion may consist of 17, 18, or 19 nucleotides, respectively, in the sequence of 20-22 contiguous nucleotides set forth in any one of SEQ ID NOs: 1-12655. For example, a guide sequence portion having 17 nucleotides in the sequence of 17 contiguous nucleotides set forth in SEQ ID NO: 9239 may consist of any one of the following nucleotide sequences (nucleotides excluded from the contiguous sequence are marked in strike-through):

SEQ ID NO: 9239 -
ACAUUCCUCACCAGCCAGUC

17 nucleotide guide sequence 1:
UUCCUCACCAGCCAGUC
(SEQ ID NO: 12656)
17 nucleotide guide sequence 2:
AUUCCUCACCAGCCAGU
(SEQ ID NO: 12657)
17 nucleotide guide sequence 3:
CAUUCCUCACCAGCCAG
(SEQ ID NO: 12658)
17 nucleotide guide sequence 4:
ACAUUCCUCACCAGCCA
(SEQ ID NO: 12659)

In embodiments of the present invention, the guide sequence portion may be greater than 22 nucleotides in length. For example, in embodiments of the present invention the guide sequence portion may be 23, 24 or 25 nucleotides in length. In such embodiments the guide sequence portion comprises: (a) 20 nucleotides in the sequence of 20 contiguous nucleotides set forth in any one of SEQ ID NOs listed in Column 2 of Table 1, (b) 21 nucleotides in the sequence of 21 contiguous nucleotides set forth in any one of SEQ ID NOs listed in Column 3 of Table 1, or (c) 22 nucleotides in the sequence of 22 contiguous nucleotides set forth in any one of SEQ ID NOs listed in Column 4 of Table 1; and additional nucleotides fully complimentary to a nucleotide or sequence of nucleotides adjacent to the 3′ end of the target sequence, 5′ end of the target sequence, or both.

In embodiments of the present invention, a CRISPR nuclease and an RNA molecule comprising a guide sequence portion form a CRISPR complex that binds to a target DNA sequence to effect cleavage of the target DNA sequence, CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule comprising a guide sequence portion of the present invention, and a tracrRNA molecule.

In embodiments of the present invention, the RNA molecule may further comprise the sequence of a tracrRNA molecule. Such embodiments may be designed as a synthetic fusion of the guide portion of the RNA molecule and the trans-activating crRNA (tracrRNA). (See Jinek (2012) Science). Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See e.g., U.S. Pat. No. 8,906,616). In embodiments of the present invention, the RNA molecule may further comprise a portion having a tracr mate sequence.

A “gene,” for the purposes of the present disclosure, includes a DNA region encoding a gene product such as RNA or protein product, including all transcribed regions such as introns and exons, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which possesses the phosphodiester bond cleaving activity. Gene modification can be achieved using a nuclease, for example a CRISPR nuclease.

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The term “nucleic acid template” refers to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1,000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position.

### Embodiments

The present disclosure provides a method for utilizing a CRISPR complex to treat, prevent or ameliorate a disease associated mutation for targeting at least one of two alleles of a gene bearing a mutation causing a disease phenotype. In some embodiments, a disease associated mutation is targeted for distinguishing/discriminating between two alleles of a gene, a first allele bearing the disease associated mutation, and the other allele not bearing the same disease associated mutation (bearing a different disease associated mutation). In some embodiments, the method further comprises the step of allele cleavage by a CRISPR nuclease. The allele cleavage is selected from the group consisting of: a double strand break and a single strand break. In some embodiments, the method further comprises the step of correction of the allele such that the corrected allele result in an expression of a functional ADA2 protein. In some embodiments, the correction is performed by homology directed repair (HDR). In some embodiments, the method further comprises the step of editing/correcting/modifying a sequence of the first allele such as to allow expression of a functional ADA2 protein. In some embodiments, the method further comprises the step of editing/correcting/modifying sequences of the two alleles such as to allow expression of a functional ADA2 protein.

According to embodiments of the present invention, there is provided an RNA molecule comprising a guide sequence portion having 17-25 nucleotides in the sequence of 20-22 contiguous nucleotides set forth in any one of SEQ ID NOs: 1-12655.

According to embodiments of the present invention, an RNA molecule may further comprise a portion having a sequence which binds to a CRISPR nuclease.

According to embodiments of the present invention, the sequence which binds to a CRISPR nuclease is a tracrRNA sequence.

According to embodiments of the present invention, an RNA molecule may further comprise a portion having a tracr mate sequence.

According to embodiments of the present invention, an RNA molecule may further comprise one or more linker portions.

According to embodiments of the present invention, an RNA molecule may be up to 500, 400, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 30 up to 30 nucleotides in length, 100 up to 300 nucleotides in length, 150 up to 300 nucleotides in length, 200 up to 300 nucleotides in length, 100 to 200 nucleotides in length, or 150 up to 250 nucleotides in length. Each possibility represents a separate embodiment.

According to some embodiments of the present invention, there is provided a composition comprising an RNA molecule comprising a guide sequence portion haying 17-25 nucleotides in the sequence of 20-22 contiguous nucleotides set forth in any one of SEQ ID NOs: 1-12655 and a CRISPR nuclease. According to some embodiments of the present invention, there is provided a composition comprising an RNA molecule comprising a guide sequence portion having 17-22 nucleotides in the sequence of 20-22 contiguous nucleotides set forth in any one of SEQ ID NOs: 1-12655 and a CRISPR nuclease.

According to embodiments of the present invention, the composition may comprise a nucleic acid template for homology-directed repair, alteration, or replacement of a target DNA sequence comprising the pathogenic mutation (e. g., allele bearing a disease-associated mutation two alleles bearing one or more disease-associated mutation, two allele bearing a disease-associated mutation).

According to embodiments of the present invention, the composition comprises a first and a second RNA molecule each comprising a guide sequence portion having 17-25 nucleotides in the sequence of 20-22 contiguous nucleotides set forth in any one of SEQ ID NOs: 1-12655, wherein the sequence of the guide sequence portion of the first RNA molecule is different from the sequence of the guide sequence portion of the second RNA molecule.

According to some embodiments of the present invention, there is provided a method for repairing/correcting a mutant ADA2 allele in a cell, the method comprising delivering to the cell a composition comprising an RNA molecule comprising a guide sequence portion having 17-25 nucleotides comprising the sequence of 20-22 contiguous nucleotides set forth in any one of SEQ ID NOs: 1-12655 and a CRISPR nuclease. According to some embodiments of the present invention, there is provided a method for repairing/correcting a mutant ADA2 allele in a cell, the method comprising delivering to the cell a composition comprising an RNA molecule comprising a guide sequence portion having 17-22 nucleotides in the sequence of 20-22 contiguous nucleotides set forth in any one of SEQ ID NOs: 1-12655 and a CRISPR nuclease.

According to some embodiments of the present invention, there is provided a method for treating DADA2, the method comprising delivering to a subject or cell obtained from a subject having DADA2 a composition comprising an RNA molecule comprising a guide sequence portion having 17-25 nucleotides comprising the sequence of 20-22 contiguous nucleotides set forth in any one of SEQ ID NOs: 1-12655 and a CRISPR nuclease. According to some embodiments of the present invention, there is provided a method for treating DADA2, the method comprising delivering to a subject or cell obtained from a subject having DADA2 a composition comprising an RNA molecule comprising a guide sequence portion having 17-22 nucleotides in the sequence of 20-22 contiguous nucleotides set forth in any one of SEQ ID NOs: 1-12655 and a CRISPR nuclease.

In a non-limiting example, an RNA molecule comprising a guide sequence is utilized to direct a CRISPR nuclease to a mutant allele and create a double-strand break (DSB) and correction/repair of the mutant allele is further performed, such as by utilizing homology directed repair (HDR), which incorporates a homologous strand as a repair template.

According to embodiments of the present invention, the CRISPR nuclease and the RNA molecule or RNA molecules are delivered to the subject and/or cells obtained from the subject substantially at the same time or at different times.

According to embodiments of the present invention, the tracrRNA is delivered to the subject and/or cells obtained from the subject substantially at the same time or at different times as the CRISPR nuclease and RNA molecule or RNA molecules.

According to embodiments of the present invention, the nucleic acid template is delivered to the subject and/or cells obtained from the subject substantially at the same time or at different times as the CRISPR nuclease and RNA molecule or RNA molecules.

According to embodiments of the present invention, the method comprises obtaining the cell with a mutant ADA2 allele from a subject with a mutant ADA2 allele and which subject is (a) homozygous for the mutant ADA2 allele, or (b) heterozygous for the mutant ADA2 allele and a second, different mutant ADA2 allele. In an embodiment, the method comprises obtaining the cell from the subject by mobilization and/or by apheresis. In an embodiment, the method comprises obtaining the cell from the subject by bone marrow aspiration.

In embodiments in which the subject/cell bears two heterozygous mutant ADA2 alleles (e.g., in case of compound heterozygous mutations), a first allele comprising a first mutation and a second allele comprising a second mutation, the correction strategies include: (a) utilizing a gRNA sequence to apply a DSB in a first allele comprising a first mutation or (b) utilizing a gRNA sequence to apply a DSB in a first allele comprising a first mutation and a second gRNA sequence to apply a DSB in a second allele comprising a second mutation.

According to some embodiments of the present invention, the RNA molecule targets a disease-causing mutation in only one of the two different mutant alleles. According to some embodiments of the present invention, the RNA molecule targets a disease-causing mutation that is common to both mutant alleles of ADA2. According to some embodiments of the present invention, the RNA molecule comprises a first guide sequence that targets a disease-causing mutation in one of the two different mutant alleles and a second guide sequence that targets a disease-causing mutation in the other of the two different mutant alleles. According to some embodiments of the present invention, the RNA molecule targets a disease-causing mutation in one of the two different mutant alleles and a second RNA molecule comprising a second guide sequence targets a disease-causing mutation in the other of the two different mutant alleles.

In embodiments where the first and second guide sequences are on two separate RNA molecules, the two separate RNA molecules may be delivered to the cells substantially at the same time or at different times.

In an embodiment of the method of correcting a mutant ADA2 allele in a cell, the cell is prestimulated prior to introducing the composition to the cell. In an embodiment, delivering the composition comprises electroporation of the cell or cells

In an embodiment of the method of correcting a mutant ADA2 allele in a cell, the method comprises culture expanding the cell to obtain cells. In an embodiment the cells are cultured the cells are cultured with: (a) one or more of: stem cell factor (SCF), IL-3, and GM-CSF; and/or (b) at least one cytokine, wherein the at least one cytokine is preferably a recombinant human cytokine.

According to some embodiments of the present invention, there is provided a modified cell obtained by the methods described herein. In an embodiment, the modified cells are obtained from culture expanding the modified cell obtained by the methods described herein. In an embodiment, the modified cells are capable of engraftment. In an embodiment, the modified cells are capable of giving rise to progeny cells. In an embodiment, the modified cells are capable of giving rise to progeny cells after engraftment. In an embodiment, the modified cells are capable of giving rise to progeny cells after an autologous engraftment. In an embodiment, the modified cells are capable of giving rise to progeny cells for at least 12 months or at least 24 months after engraftment.

In an embodiment the modified cell or cells are hematopoietic stem cells and/or progenitor cells (HSPCs), preferably wherein the modified cell or cells are CD34+ hematopoietic stem cells. In an embodiment the modified cell or cells are bone marrow cells or peripheral mononucleated cells (PMCs).

According to some embodiments of the present invention, there is provided a composition/kit comprising the modified cells and a pharmaceutically acceptable carrier. Also provided is an in vitro or ex vivo method of preparing this composition, comprising mixing the modified cells of the invention with the pharmaceutically acceptable carrier.

In some embodiments, the method further comprises, utilizing a nucleic acid template for homology-directed repair, alteration, or replacement of the entire exon or a portion of the exon or multiple exons or the entire open reading frame of a gene, or the entire gene.

According to some embodiments of the present invention, there is provided a method of treating a subject afflicted with Adenosine deaminase 2 (ADA2) deficiency, comprising administration of a therapeutically effective amount of the modified cells the invention, the composition comprising the modified cells of the invention and a pharmaceutically acceptable carrier, the cells obtained by the methods of the invention, or a composition prepared by the in vitro or ex vivo method of preparing the composition comprising the modified cells of the invention and a pharmaceutically acceptable carrier.

According to some embodiments of the present invention, there is provided use of an RNA molecule of the invention for correcting a mutant ADA2 allele in a cell.

According to some embodiments of the present invention, there is provided use of a composition comprising an RNA molecule comprising a guide sequence portion having 17-25 (e.g., 17-20, 17-21, 17-22, 17-23, 17-24, 20-24, etc.) nucleotides comprising the sequence of 20-22 contiguous nucleotides set forth in any one of SEQ ID NOs: 1-12655 and a CRISPR nuclease repairing/correcting/modifying a mutant ADA2 allele in a cell, comprising delivering to the cell the RNA molecule comprising a guide sequence portion having 17-25 (e.g., 17-20, 17-21, 17-22, 17-23, 17-24, 20-24, etc.) nucleotides in the sequence of 20-22 contiguous nucleotides set forth in any one of SEQ ID NOs: 1-12655 and the CRISPR nuclease.

According to embodiments of the present invention, there is provided a medicament comprising an RNA molecule comprising a guide sequence portion having 17-25 (e.g., 17-20, 17-21, 17-22, 17-23, 17-24, 20-24, etc.) nucleotides comprising the sequence of 20-22 contiguous nucleotides set forth in any one of SEQ ID NOs: 1-12655 and a CRISPR nuclease for use in repairing/correcting/modifying a mutant ADA2 allele in a cell, wherein the medicament is administered by delivering to the cell the composition comprising an RNA molecule comprising a guide sequence portion having 17-25 (e.g., 17-20, 17-21, 17-22, 17-23, 17-24, 20-24, etc.) nucleotides in the sequence of 20-22 contiguous nucleotides set forth in any one of SEQ ID NOs: 1-12655 and a CRISPR nuclease.

According to some embodiments of the present invention, there is provided use of a composition comprising an RNA molecule comprising a guide sequence portion having 17-25 (e.g., 17-20, 17-21, 17-22, 17-23, 17-24, 20-24, etc.) nucleotides comprising the sequence of 20-22 contiguous nucleotides set forth in any one of SEQ ID NOs: 1-12655 and a CRISPR nuclease for treating ameliorating or preventing DADA2, comprising delivering to a subject having or at risk of having DADA2 the composition of comprising an RNA molecule comprising a guide sequence portion having 17-25 (e.g., 17-20, 17-21, 17-22, 17-23, 17-24, 20-24, etc.) nucleotides comprising the sequence of 20-22 contiguous nucleotides set forth in any one of SEQ ID NOs: 1-12655 and a CRISPR nuclease.

According to some embodiments of the present invention, there is provided a medicament comprising the composition comprising an RNA molecule comprising a guide sequence portion having 17-25 (e.g., 17-20, 17-21, 17-22, 17-23, 17-24, 20-24, etc.) nucleotides comprising the sequence of 20-22 contiguous nucleotides set forth in any one of SEQ ID NOs: 1-12655 and a CRISPR nuclease for use in treating ameliorating or preventing DADA2, wherein the medicament is administered by delivering to a subject having or at risk of having DADA2: the composition comprising an RNA molecule comprising a guide sequence portion having 17-25 (e.g., 17-20, 17-21, 17-22, 17-23, 17-24, 20-24, etc.) nucleotides comprising the sequence of 20-22 contiguous nucleotides set forth in any one of SEQ NOs: 1-12655 and a CRISPR nuclease. In some embodiments, the medicament further comprises, a nucleic acid template for homology-directed repair, alteration, or replacement of at least a portion of a target gene.

According to some embodiments of the present invention, there is provided a kit for repairing/correcting/modifying a mutant DADA2 allele in a cell, comprising an RNA molecule comprising a guide sequence portion having 17-25 (e.g., 17-20, 17-21, 17-22, 17-23, 17-24, 20-24, etc.) nucleotides comprising the sequence of 20-22 contiguous nucleotides set forth in any one of SEQ ID NOs: 1-12655, a CRISPR nuclease, and/or a tracrRNA molecule; and instructions for delivering the RNA molecule; CRISPR nuclease, and/or the tracrRNA to the cell. In some embodiments, the kit further comprises, a nucleic acid template for homology-directed repair, alteration, or replacement of of at least a portion of a target gene.

According to some embodiments of the present invention, there is provided a kit for treating DADA2 in a subject, comprising an RNA molecule comprising a guide sequence portion having 17-25 (e.g., 17-20, 17-21, 17-22, 17-23, 17-24, 20-24, etc.) nucleotides comprising the sequence of 20-22 contiguous nucleotides set forth in any one of SEQ ID NOs: 1-12655, a CRISPR nuclease, and/or a tracrRNA molecule; and instructions for delivering the RNA molecule; CRISPR nuclease, and/or the tracrRNA to a subject having or at risk of having DADA2. in some embodiments, the kit further comprises, a nucleic acid template for homology-directed repair, alteration, or replacement of at least a portion of a target gene.

In embodiments of the present invention, the RNA molecule comprises a guide sequence portion having 17-25 (e.g., 17-20, 17-21, 17-22, 17-23, 17-24, 20-24, etc.) nucleotides comprising the sequence of 20-22 contiguous nucleotides set forth in any one of SEQ ID NOs: 1-12655. In embodiments of the present invention, the RNA molecule comprises a guide sequence portion having 17-22 nucleotides in the sequence of 20-22 contiguous nucleotides set forth in any one of SEQ ID NOs: 1-12655.

The compositions and methods of the present disclosure may be utilized for treating, preventing, ameliorating, or slowing progression of DADA2.

In some embodiments, the method of repairing/correcting a mutant allele further comprises enhancing activity of the functional protein such as by providing a protein/peptide, a nucleic acid encoding a protein/peptide, or a small molecule such as a chemical compound, capable of activating/enhancing activity of the functional protein.

According to some embodiments, the present disclosure provides an RNA sequence (‘RNA molecule’) which binds to/associates with and/or directs the RNA guided DNA nuclease e.g., CRISPR nuclease to a sequence comprising at least one nucleotide which differs between a first mutant allele and a second mutant allele (e.g., different disease associated mutation) of a gene of interest.

In some embodiments, the method comprises the steps of: contacting a first mutant allele (i.e., a first of two mutant alleles bearing the same or a different disease associated mutation) of a gene of interest with an allele-specific RNA molecule and a CRISPR nuclease e.g., a Cas9 protein, wherein the allele-specific RNA molecule and the CRISPR nuclease e.g., Cas9 associate with a nucleotide sequence of the first mutant allele of the gene of interest which differs by at least one nucleotide from a nucleotide sequence of a second allele of the gene of interest (i.e., a second mutant allele of the two mutant alleles), thereby modifying the first mutant allele.

In some embodiments, the allele-specific RNA molecule and a CRISPR nuclease are introduced to a cell encoding the gene of interest. In some embodiments, the cell encoding the gene of interest is in a mammalian subject. In some embodiments, the cell encoding the gene of interest is an eukaryotic cell. In some embodiments, the cell encoding the gene of interest is a mammalian cell.

In some embodiments, a nucleic acid template is further introduced to the cell encoding the gene of interest for homology-directed repair, alteration, or replacement of a target sequence of the gene of interest to correct/repair the gene of interest such as to express a functional protein.

In some embodiments, the mutant allele is an allele of the ADA2 gene. In some embodiments, a disease-causing mutation within a mutated ADA2 allele is targeted.

In some embodiments, the method is utilized for treating a subject having a disease phenotype resulting from the ADA2 gene. In such embodiments, the method results in improvement, amelioration or prevention of the disease phenotype.

Embodiments referred to above refer to a CRISPR nuclease, RNA molecule(s), and optionally tracrRNA being effective in a subject or cells at the same time. The CRISPR, RNA molecule(s), and optionally tracrRNA can be delivered substantially at the same time or can be delivered at different times but have effect at the same time. For example, this includes delivering the CRISPR nuclease to the subject or cells before the RNA molecule and/or tracr RNA is substantially extant in the subject or cells.

In one embodiment, the cell is a stem cell. In one embodiment, the cell is an embryonic stem cell. In some embodiment, the stem cell is a hematopoietic stem/progenitor cell (HSC). As used herein, the term HSC refers to both hematopoietic stem cells and hematopoietic stem progenitor cells. Non-limiting examples of stem cells include hone marrow cells, myeloid progenitor cells, a multipotent progenitor cells, a lineage restricted progenitor cells.

As used herein, “progenitor cell” refers to a lineage cell that is derived from stem cell and retains mitotic capacity and multipotency (e.g., can differentiate or develop into more than one but not all types of mature lineage of cell). As used herein “hematopoiesis” or “hemopoiesis” refers to the formation and development of various types of blood cells (e.g., red blood cells, megakaryocytes, myeloid cells (e.g., monocytes, macrophages and neutrophil), and lymphocytes) and other formed elements in the body (e.g., in the bone marrow).

### Recessive Genetic Disorders

One of skill in the art will appreciate that all subjects with any type of recessive genetic disorder may be subjected to the methods described herein. In one embodiment, the present invention may be used to target a gene involved in, associated with, or causative of a recessive genetic disorders such as, for example DADA2. In some embodiments, the recessive genetic disorder is DADA2. In some embodiments, the target gene is the ADA2 gene (Entrez Gene, gene ID No: 51816). DADA2 is a recessive genetic condition caused by mutations in the CECR1(ADA2) gene that prevent it from correctly encoding the enzyme Adenosine Deaminase 2 (ADA2). Some patients are homozygous, while others are compound heterozygous and have two different mutations.

### CRISPR Nucleases and PAM Recognition

In some embodiments, the sequence specific nuclease is selected from CRISPR nucleases, or a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to the sequence comprising the mutation. In some embodiments, the CRISPR complex does not further comprise a tracrRNA. In a non-limiting example, in which the RNA guided DNA nuclease is a CRISPR protein, the at least one nucleotide which differs between a target allele (e.g., bearing a pathogenic mutation) and the other allele (e.g., bearing a different or the same pathogenic mutation) may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

In embodiments of the present invention, a type II CRISPR system utilizes a mature crRNA:tracrRNA complex directs a CRISPR nuclease, e.g. Cas9, to the target DNA via Watson-Crick base-pairing between the crRNA and the protospacer on the target DNA next to the protospacer adjacent motif (PAM), an additional requirement for target recognition. The CRISPR nuclease then mediates cleavage of target DNA to create a double-stranded break within the protospacer. A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NNNNGATT for Neisseria meningitidis (NmCas9); or TTTV for Cpf1. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015-0211023, incorporated herein by reference.

CRISPR systems that may be used in the practice of the invention vary greatly, CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas1 Od, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10 Csx16, CsaX, Csx3, Csz1, Csx15, Csf1, Csf2, Csf3, Csf4, and Cu1966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponerma denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., spCas9 D1135E variant, spCas9 VQR variant, spCas9 EQR variant, or spCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al. (2015) Cell.).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseudouridine, “beta, D-galactosylquenosine”, 2′-O-methylguanosine, inosine, N6-isopentenyladenosine, 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylquenosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N-((9-beta-D-ribofuranosylpurine-6-y)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, 5-methyluridine, N-((9-beta-D-ribofuranosylpurine-6-yl)-carbamoyl)threonine, 2′-O-methyl-5-methyluridine, 2′-O-methyluridine, wybutosine, “3-(3-amino-3-carboxy-propyl)uridine, (acp3)u”, 2′-0-methyl (M), 3′-phosphorothioate (MS), 3′-thioPACE (MSP), pseudouridine, or 1-methyl pseudo-uridine. Each possibility represents a separate embodiment of the present invention.

### Guide Sequences which Specifically Target a Mutant Allele

Utilizing a 24 base pair target window for targeting a imitation in a gene would require hundreds of thousands of guide sequences. Any given guide sequence when utilized to target a sequence may result in degradation of the guide sequence, limited activity, no activity, or off-target effects. Accordingly, suitable guide sequences are necessary for targeting a given gene. By the present invention, a novel set of guide sequences have been identified for repairing/correcting/modifying a mutant allele or two mutant alleles of ADA2 gene to express a functional ADA2 protein and treat DADA2.

The present disclosure provides guide sequences capable of specifically targeting a first mutant allele while leaving a second mutant allele unmodified. The guide sequences of the present invention are designed to, and are most likely to, specifically differentiate between a first mutant allele and a second mutant allele. Of all possible guide sequences which target a first mutant allele desired to be edited/corrected/modified/repaired, the specific guide sequences disclosed herein are specifically effective to function with the disclosed embodiments.

The present disclosure also provides guide sequences capable of specifically targeting a first and a second mutant alleles. Of all possible guide sequences which target the first and second mutant alleles desired to be edited/corrected/modified/repaired, the specific guide sequences disclosed herein are specifically effective to function with the disclosed embodiments.

Briefly, the guide sequences may have properties as follows: target a mutant allele using an RNA molecule which targets a founder or common pathogenic mutations for the disease/gene

Guide sequences of the present invention also may target: (1) have a guanine-cytosine content of greater than 30% and less than 85%; (2) have no repeat of 4 or more thymine/uracil or 8 or more guanine, cytosine, or adenine; (3) having no off-target identified by off-target analysis.

Guide sequences of the present invention may satisfy any one of the above criteria and are most likely to differentiate between a mutant allele from its corresponding functional allele.

### Examples of RNA Guide Sequences which Specifically Target Mutant Allele or Alleles of ADA2 Gene

Although a large number of guide sequences can be designed to target a mutant allele, the nucleotide sequences described in SEQ ID NOs: 1-12655 were specifically selected to effectively implement the methods set forth herein and to effectively discriminate between alleles.

The nucleotide sequences described in SEQ ID NOs: 1-12655 are guide sequences designed for use as described in the embodiments above to associate with sequences of a mutated ADA2 allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments the RNA molecule targets an ADA2 gene mutation as shown in Table 1 below. The ADA2 gene mutation details are indicated in the 1^{st} column. Columns 2-4 describe the sequences of guides targeting these mutations by reference to a SEQ ID NO in the Sequence Listing.

TABLE 1
ADA2 gene mutations and sequences of guides targeting these mutations

| | SEQ ID NOs | SEQ ID NOs | SEQ ID NOs |
|---|---|---|---|
| | of 20 | of 21 | of 22 |
| Mutation | base guides | base guides | base guides |
| 22:17181488_T_G | 1-46 | 47-94 | 95-144 |
| 22:17181518_A_G | 145-190 | 191-238 | 239-288 |
| 22:17181552_C_G | 289-334 | 335-382 | 383-432 |
| 22:17181554_C_G | 292, 300, | 341, 360, | 389, 396, |
| | 313, 319, | 362, 367, | 410-411, |
| | 433-474 | 475-518 | 519-564 |
| 22:17181814_A_G | 565-610 | 611-658 | 659-708 |
| 22:17181839_G_T | 709-754 | 755-802 | 803-852 |
| 22:17181877_A_G | 853-898 | 899-946 | 947-996 |
| 22:17181889_A_T | 997-1042 | 1043-1090 | 1091-1140 |
| 22:17181904_T_C | 1141-1186 | 1187-1234 | 1235-1284 |
| 22:17181909_C_G | 1285-1330 | 1331-1378 | 1379-1428 |
| 22:17181914_C_A | 1429-1474 | 1475-1522 | 1523-1572 |
| 22:17181993_G_C | 1573-1618 | 1619-1666 | 1667-1716 |
| 22:17182620_C_T | 1717-1762 | 1763-1810 | 1811-1860 |
| 22:17182671_T_C | 1861-1906 | 1907-1954 | 1955-2004 |
| 22:17182696_C_T | 2005-2050 | 2051-2098 | 2099-2148 |
| 22:17182725_T_A | 2149-2194 | 2195-2242 | 2243-2292 |
| 22:17182733_G_T | 2293-2338 | 2339-2386 | 2387-2436 |
| 22:17183873_CT_C | 2437-2482 | 2483-2530 | 2531-2580 |
| 22:17188342_T_C | 2581-2626 | 2627-2674 | 2675-2724 |
| 22:17188348_C_T | 2725-2770 | 2771-2818 | 2819-2868 |
| 22:17188351_C_T | 2728, 2740, | 2771, 2785, | 2833, 2854, |
| | 2869-2912 | 2913-2958 | 2959-3006 |
| 22:17188357_A_G | 3007-3052 | 3053-3100 | 3101-3150 |
| 22:17188375_C_T | 3151-3196 | 3197-3244 | 3245-3294, |
| 22:17188389_G_A | 3295-3340 | 3341-3388 | 3389-3438 |
| 22:17188402_C_A | 3439-3484 | 3485-3532 | 3533-3582 |
| 22:17188413_T_C | 3583-3628 | 3629-3676 | 3677-3726 |
| 22:17188415_ATG_ACA | 3590, 3619, | 3637, 3654, | 3703, 3709, |
| | 3727-3770 | 3771-3816 | 3817-3864 |
| 22:17188416_TG_CA | 3619, | 3637, | 3703, |
| | 3727-3756, | 3771-3800, | 3817-3848, |
| | 3758-3770, | 3802-3816, | 3850-3864, |
| | 3865-3866 | 3867-3868 | 3869-3870 |
| 22:17188436_C_G | 3871-3916 | 3917-3964 | 3965-4014 |
| 22:17188438_C_T | 3878, 3888, | 3924, 3929, | 3978, 3982, |
| | 3909, 3911, | 3933, 3957, | 4003, 4006, |
| | 4015-4056 | 4057-4100 | 4101-4146 |
| 22:17188448_C_T | 4147-4192 | 4193-4240 | 4241-4290 |
| 22:17188449_T_C | 4156, 4160, | 4206, 4212, | 4257, 4262, |
| | 4164, 4173, | 4220, 4222, | 4272, 4276, |
| | 4175, 4182, | 4230, 4237, | 4280, 4287, |
| | 4291-4330 | 4331-4372 | 4373-4416 |
| 22:17189939_T_C | 4417-4462 | 4463-4510 | 4511-4560 |
| 22:17189952_C_T | 4561-4606 | 4607-4654 | 4655-4704 |
| 22:17189964_G_A | 4705-4750 | 4751-4798 | 4799-4848 |
| 22:17189979_C_G | 4849-4894 | 4895-4942 | 4943-4992 |
| 22:17189982_A_C | 4860, 4864, | 4897, 4911, | 4952, 4982, |
| | 4993-5036 | 5037-5082 | 5083-5130 |
| 22:17189987_C_T | 5131-5176 | 5177-5224 | 5225-5274 |
| 22:17189998_G_A | 5275-5320 | 5321-5368 | 5369-5418 |
| 22:17190034_T_C | 5419-5464 | 5465-5512 | 5513-5562 |
| 22:17191692_G_A | 5563-5608 | 5609-5656 | 5657-5706 |
| 22:17191773_C_G | 5707-5752 | 5753-5800 | 5801-5850 |
| 22:17191780_C_T | 5851-5896 | 5897-5944 | 5945-5994 |
| 22:17191781_GTC_GGG | 5851, | 5897, 5926, | 5978-5979, |
| | 5859-5860, | 5931, 5934, | 5982, 5989, |
| | 5886, | 6037-6080 | 6081-6126 |
| | 5995-6036 | | |
| 22:17203563_C_T | 6127-6172 | 6173-6220 | 6221-6270 |
| 22:17203564_G_A | 6129, 6133, | 6174, 6176, | 6221-6222, |
| | 6140, | 6180, 6187, | 6235, 6249, |
| | 6152-6153, | 6200, 6212, | 6261, 6269, |
| | 6157, | 6311-6352 | 6353-6396 |
| | 6271-6310 | | |
| 22:17203570_A_G | 6397-6442 | 6443-6490 | 6491-6540 |
| 22:17203576_G_A | 6541-6586 | 6587-6634 | 6635-6684 |
| 22:17203587_C_T | 6685-6730 | 6731-6778 | 6779-6828 |
| 22:17203588_A_C | 6688, 6690, | 6734, 6742, | 6796, 6799, |
| | 6696, 6700, | 6750, 6760, | 6809, 6815, |
| | 6712, 6722, | 6766, 6770, | 6819, 6822, |
| | 6829-6868 | 6869-6910 | 6911-6954 |
| 22:17203604_C_T | 6955-7000 | 7001-7048 | 7049-7098 |
| 22:17203607_CGTA_C | 6700, 6974, | 6746, 7041, | 7057, |
| | 7099-7142 | 7143-7188 | 7189-7237 |
| 22:17203628_G_A | 7238-7283 | 7284-7331 | 7332-7381 |
| 22:17203651_GGTGC_G | 7382-7427 | 7298, | 7333, 7347, |
| | | 7428-7474 | 7475-7522 |
| 22:17203655_CGTA_C | 7385, 7395, | 7431, 7433, | 7476, 7481, |
| | 7400, 7410, | 7435, 7457, | 7487, 7505, |
| | 7523-7564 | 7565-7608 | 7609-7654 |
| 22:17203738_G_A | 7655-7700 | 7701-7748 | 7749-7798 |
| 22:17203753_A_G | 7799-7844 | 7845-7892 | 7893-7942 |
| 22:17207070_C_T | 7943-7988 | 7989-8036 | 8037-8086 |
| 22:17207080_A_G | 8087-8132 | 8133-8180 | 8181-8230 |
| 22:17207107_C_T | 8231-8276 | 8277-8324 | 8325-8374 |
| 22:17207152_C_T | 8375-8420 | 8421-8468 | 8469-8518 |
| 22:17207179_T_G | 8519-8564 | 8565-8612 | 8613-8662 |
| 22:17207185_A_C | 8663-8708 | 8709-8756 | 8757-8806 |
| 22:17207219_GC_G | 8807-8852 | 8853-8900 | 8901-8950 |
| 22:17207225_A_G | 8951-8996 | 8997-9044 | 9045-9094 |
| 22:17207236_C_T | 9095-9140 | 9141-9188 | 9189-9238 |
| 22:17207251_A_G | 9239-9284 | 9285-9332 | 9333-9382 |
| 22:17207254_G_A | 9255, 9259, | 9285, 9323, | 9360-9361, |
| | 9383-9426 | 9427-9472 | 9473-9520 |
| 22:17207277_G_C | 9521-9566 | 9567-9614 | 9615-9664 |
| 22:17207287_G_T | 9665-9710 | 9711-9758 | 9759-9808 |
| 22:17209400_A_G | 9809-9854 | 9855-9902 | 9903-9952 |
| 22:17209484_G_A | 9953-9998 | 9999-10046 | 10047-10096 |
| 22:17209492_C_A | 10097-10142 | 10143-10190 | 10191-10240 |
| 22:17209512_CCTT_C | 10241-10286 | 10287-10334 | 10335-10383 |
| 22:17209531_CCG_CGC | 10384-10403 | 10404-10423 | 10424-10443 |
| 22:17209533_GCCCCCCC_G | 10386, 10388, | 10405, 10409, | 10425, 10429, |
| | 10400, | 10420, | 10440, |
| | 10444-10486 | 10487-10531 | 10532-10578 |
| 22:17209533_GC_G | 10271, | 10319-10322, | 10369-10371, |
| | 10273-10275, | 10404-10405, | 10424-10425, |
| | 10384, 10386, | 10409, 10420, | 10429, 10440, |
| | 10388, 10400, | 10504, 10508, | 10549, 10568, |
| | 10464, 10486, | 10615-10652 | 10653-10693 |
| | 10579-10614 | | |
| 22:17209533_G_A | 10384, 10386, | 10404-10405, | 10424-10425, |
| | 10388, 10400, | 10409, 10420, | 10429, 10440, |
| | 10464, | 10504, 10615, | 10568, 10657, |
| | 10579-10580, | 10632, | 10672, |
| | 10694-10706 | 10707-10719 | 10720-10732 |
| 22:17209533_G_C | 10384, 10386, | 10404-10405, | 10424-10425, |
| | 10388, 10400, | 10409, 10420, | 10429, 10440, |
| | 10464, | 10504, 10615, | 10568, 10657, |
| | 10579-10580, | 10632, | 10672, |
| | 10733-10739 | 10740-10746 | 10747-10753 |
| 22:17209535_C_T | 10386, 10464, | 10420, 10504, | 10429, 10549, |
| | 10486, | 10508, 10615, | 10568, 10654, |
| | 10579-10580, | 10618, 10632, | 10657, |
| | 10595, 10612, | 10652, | 10671-10672, |
| | 10754-10792 | 10793-10833 | 10834-10876 |
| 22:17209538_C_A | 10445, 10466, | 10510, 10524, | 10570, 10577, |
| | 10605-10606, | 10642-10643, | 10671, 10683, |
| | 10612-10613, | 10649, 10652, | 10690, 10693, |
| | 10756, | 10794, | 10835, |
| | 10877-10915 | 10916-10956 | 10957-10999 |
| 22:17209538_C_G | 10445, 10466, | 10510, 10524, | 10570, 10577, |
| | 10605-10606, | 10642-10643, | 10671, 10683, |
| | 10612-10613, | 10649, 10652, | 10690, 10693, |
| | 10756, 10879, | 10794, 10917, | 10835, 10981, |
| | 11000-11037 | 11038-11077 | 11078-11119 |
| 22:17209539_C_A | 10445, 10466, | 10510, 10524, | 10566, 10570, |
| | 10479, | 10530, | 10577, |
| | 10605-10607, | 10642-10643, | 10683-10684, |
| | 10613, 10879, | 10649-10650, | 10690, 10693, |
| | 11120-11157 | 10917, | 10981, |
| | | 11158-11197 | 11198-11239 |
| 22:17209539_C_T | 10445, 10466, | 10510, 10524, | 10566, 10570, |
| | 10479, | 10530, | 10577, |
| | 10605-10607, | 10642-10643, | 10683-10684, |
| | 10613, 10879, | 10649-10650, | 10690, |
| | 11240-11277 | 10917, | 10693, 10981, |
| | | 11278-11317 | 11318-11359 |
| 22:17209563_AC_A | 11360-11405 | 11406-11453 | 11454-11503 |
| 22:17209574_G_A | 11504-11549 | 11550-11597 | 11598-11647 |
| 22:17209578_G_A | 11648-11693 | 11694-11741 | 11742-11791 |
| 22:17209595_A_G | 11792-11837 | 11838-11885 | 11886-11935 |
| 22:17209599_C_A | 11936-11981 | 11982-12029 | 12030-12079 |
| 22:17209605_C_A | 12080-12125 | 12126-12173 | 12174-12223 |
| 22:17209652_C_T | 12224-12269 | 12270-12317 | 12318-12367 |
| 22:17209676_A_G | 12368-12413 | 12414-12461 | 12462-12511 |
| 22:17209684_G_C | 12512-12557 | 12558-12605 | 12606-12655 |

Strategies for HDR repair of a pathogenic mutation associated with DADA2 may involve a guide sequence targeting the pathogenic mutation itself to mediate a DSB in proximity to the mutation. The strategies may further include a sequence repair/correction step by utilizing a donor/template sequence that (e.g., a single-stranded donor oligonucleotides (ssODN), double-stranded Donor (PCR product), Minicircle or virus (rAAV or Lentivirus)).

In an exemplary strategy, a mutant allele bearing a founder mutation such as, rs202134424 (c.139G>A, p.Gly47Arg), rs200930463 (c.140G>C, p.Gly47Ala), rs77563738 (c.506G>A, p.Arg169Gln), rs376785840 (c.1358A>G, p.Tyr453Cys), rs148936893 (c. 752C>T, p.Pro251Leu), rs775440641 (c,1078A>G, p.Thr360Ala) or any other mutations as indicated in table 1, is targeted by guide sequences designed to target the founder mutation itself such as guide sequences comprising a nucleotide sequence as set forth in SEQ ID Nos: 1-12655.

### Delivery to Cells

The RNA molecule compositions described herein may be delivered to a target cell by any suitable means. RNA molecule compositions of the present invention may be targeted to any cell which contains and/or expresses a mutated allele, including any mammalian or plant cell. For example, in one embodiment the RNA molecule specifically targets a mutated ADA2 allele and the target cell is an HSC. The delivery to the cell may be performed in-vitro, ex-vivo, or in-vivo. Further, the nucleic acid compositions described herein may be delivered as one or more of DNA molecules, RNA molecules, Ribonucleoproteins (RNP), nucleic acid vectors, or any combination thereof.

In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3′ thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992) Science 256:808-813; Nabel & Felgner (1993) TIBTECH 11:211-217; Mitani & Caskey (1993) TIBTECH 11:162-166; Dillon (1993) TIBTECH 11:167-175; Miller (1992) Nature 357:455-460; Van Brunt (1988) Biotechnology 6(10):1149-1154; Vigne (1995) Restorative Neurology and Neuroscience 8:35-36; Kremer & Perricaudet (1995) British Medical Bulletin 51(1):31-44; Haddada et al, (1995) in Current Topics in Microbiology and immunology Doerfler and Bohm (eds.); and Yu et al. (1994) Gene Therapy 1:13-26.

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboium meliloti, Mesorhizobium loti, tobacco mosaic virus, potato virus X, cauliflower mosaic virus and cassava vein mosaic virus). (See, e.g., Chung et al. (2006) Trends Plant Sci. 11(1):1-4). Sonoporation using, e.g., the Sonitron 2000 system (Rich-Mar), can also be used for delivery of nucleic acids. Cationic-lipid mediated delivery of proteins and/or nucleic acids is also contemplated as an in vivo or in vitro delivery method. (See Zuris et al. (2015) Nat. Biotechnol. 33(1):73-80; see also Coelho et al. (2013) N. Engl. J. Med. 369, 819-829; Judge et al. (2006) Mol. Ther. 13, 494-505; and Basha et al. (2011) Mol. Ther. 19, 2186-2200).

Additional exemplary nucleic acid delivery systems include those provided by Amaxa® Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam™, Lipofectin™ and Lipofectamine™ RNAiMAX). Cationic and neutral lipids that are suitable for efficient receptor-recognition lipofection of polynucleotides include those of Feigner, WO 91/17424, WO 91/16024. Delivery can be to cells (ex vivo administration) or target tissues (in vivo administration).

The preparation of lipid:nucleic acid complexes, including targeted liposomes such as immunolipid complexes, is well known to one of skill in the art (See, e.g., Crystal (1995) Science 270: 404-410; Blaese et al. (1995) Cancer Gene Ther. 2:291-297; Behr et al. (1994) Bioconjugate Chem. 5:382-389; Remy et al. (1994) Bioconjugate Chem. 5:647-654; Gao et al. (1995) Gene Therapy 2:710-722; Ahmad et al. (1992) Cancer Res. 52:4817-4820; U.S. Pat. Nos. 4,186,183, 4,217,344, 4;235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al (2009) Nature Biotechnology 27(7):643).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992) J. Virol. 66:2731-2739; Johann et al. (1992) J. Virol. 66:1635-1640; Sommerfelt et al. (1990) Virol. 176:38-59; Wilson et al. (1989) J. Virol. 63:2374-2378; Miller et al. (1991) J. Virol. 65:2220-2224: PCT/US94/05700).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al. (1995) Blood 85:3048-305; Kohn et al. (1995) Nat. Med. 1:1017-102; Malech et al. (1997) PNAS 94:22 12133-12138). PA317/pLASN was the first therapeutic vector used in a gene therapy trial. (Blaese et al. (1995). Transduction efficiencies of 50% or greater have been observed for MFG-S packaged vectors. (Ellem et al. (1997) Immunol immunother. 44(1):10-20; Dranoff et al. (1997) Hum. Gene Ther. 1:111-2).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,5541.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) Proc. Natl. Acad. Sci. USA 92:9747-9751, reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney et al. (1994) Culture of Animal Cells, A Manual of Basic Technique, 3rd ed, and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO-S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), perC6 cells, any plant cell (differentiated or undifferentiated), as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., J. Exp. Med. 176:1693-1702 (1992)).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (as a non-limiting example see Inaba et al. (1992) Exp. Med. 176:1693-1702). Stem cells that have been modified may also be used in some embodiments.

Any one of the RNA molecule compositions described herein is suitable for genome editing in either mitotic cells or post-mitotic cells or any cell which is not actively dividing, e.g., arrested cells.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of transgenes into immune cells (e.g., T-cells) include non-integrating lentivirus vectors. See, e.g., U.S. Patent Publication No. 2009-0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

In accordance with some embodiments, there is provided an RNA molecule which binds to/associates with and/or directs the RNA guided DNA nuclease (E. G., CRISPR nuclease) to a sequence comprising a mutation in one or both alleles of a gene of interest

The disclosed compositions and methods may also be used in the manufacture of a medicament for treating dominant genetic disorders in a patient.

### DNA Repair by Homologous Recombination

The term “homology-directed repair” or “HDR” refers to a mechanism for repairing DNA damage in cells, for example, during repair of double-stranded and single-stranded breaks in DNA. HDR requires nucleotide sequence homology and uses a “nucleic acid template” (nucleic acid template or donor template used interchangeably herein) to repair the sequence where the double-stranded or single break occurred (e.g., DNA target sequence). This results in the transfer of genetic information from, for example, the nucleic acid template to the DNA target sequence. HDR may result in alteration of the DNA target sequence (e.g., insertion, deletion, mutation) if the nucleic acid template sequence differs from the DNA target sequence and part or all of the nucleic acid template polynucleotide or oligonucleotide is incorporated into the DNA target sequence. In some embodiments, an entire nucleic acid template polynucleotide, a portion of the nucleic acid template polynucleotide, or a copy of the nucleic acid template is integrated at the site of the DNA target sequence.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length (or any integer value there between or there above), preferably between about 100 and 1000 nucleotides in length (or any integer there between), more preferably between about 200 and 500 nucleotides in length. A nucleic acid template may be a single stranded nucleic acid, a double stranded nucleic acid. In some embodiment, the nucleic acid template comprises a nucleotide sequence, e.g., of one or more nucleotides, that corresponds to wild type sequence of the target nucleic acid, e.g., of the target position. In some embodiment, the nucleic acid template comprises a ribonucleotide sequence, e.g., of one or more ribonucleotides, that corresponds to wild type sequence of the target nucleic acid, e.g., of the target position. In some embodiment, the nucleic acid template comprises modified ribonucleotides.

Insertion of an exogenous sequence (also called a “donor sequence,” donor template” or “donor”), for example, for correction of a mutant gene or for increased expression of a wild-type gene can also be carried out. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded and/or double-stranded and can be introduced into a cell in linear or circular form. See, e.g., U.S. Patent Publication Nos. 20100047805; 201 10281361; and 20110207221. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

Accordingly embodiments of the present invention using a donor DNA template for HDR may use a DNA or RNA, single-stranded and/or double-stranded and can be introduced into a cell in linear or circular form. In an embodiment of the present invention using: (1) an RNA molecule comprising a guide sequence to affect a double strand break in a gene prior to HDR and (2) a donor RNA template for HDR, the RNA molecule comprising the guide sequence is a first RNA molecule and the donor RNA template is a separate RNA molecule. In an embodiment, the RNA molecule comprising the guide sequence also comprises the donor RNA template.

A donor sequence may also be air oligonucleotide and be used for gene correction or targeted alteration of an endogenous sequence. The oligonucleotide may be introduced to the cell on a vector, may be electroporated into the cell, or may be introduced via other methods known in the art. The oligonucleotide can be used to ‘correct’ a mutated sequence in an endogenous gene (e.g., the sickle mutation in beta globin), or may be used to insert sequences with a desired purpose into an endogenous locus.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV).

The donor is generally inserted so that its expression is driven by the endogenous promoter at the integration site, namely the promoter that drives expression of the endogenous gene into which the donor is inserted. However, it will be apparent that the donor may comprise a promoter and/or enhancer, for example a constitutive promoter or an inducible or tissue specific promoter.

The donor molecule may be inserted into an endogenous gene such that all, some or none of the endogenous gene is expressed. For example, a transgene as described herein may be inserted into an endogenous locus such that some (N-terminal and/or C-terminal to the transgene) or none of the endogenous sequences are expressed, for example as a fusion with the transgene. In other embodiments, the transgene (e.g., with or without additional coding sequences such as for the endogenous gene) is integrated into any endogenous locus, for example a safe-harbor locus, for example a CCR5 gene, a CXCR4 gene, a PPP1R12c (also known as AAVS1) gene, an albumin gene or a Rosa gene. See, e.g., U.S. Pat. Nos. 7,951,925 and 8,110,379; U.S. Publication Nos. 20080159996; 201000218264; 20100291048; 20120017290; 20110265198; 20130137104; 20130122591; 20130177983 and 20130177960 and U.S. Provisional Application No. 61/823,689).

When endogenous sequences (endogenous or part of the transgene) are expressed with the transgene, the endogenous sequences may be full-length sequences (wild-type or mutant) or partial sequences. Preferably the endogenous sequences are functional. Non-limiting examples of the function of these full length or partial sequences include increasing the serum half-life of the polypeptide expressed by the transgene (e.g., therapeutic gene) and/or acting as a carrier.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

In certain embodiments, the donor molecule comprises a sequence selected from the group consisting of a gene encoding a protein (e.g., a coding sequence encoding a protein that is lacking in the cell or in the individual or an alternate version of a gene encoding a protein), a regulatory sequence and/or a sequence that encodes a structural nucleic acid such as microRNA or siRNA.

### General

For the foregoing embodiments, each embodiment disclosed herein is contemplated as being applicable to each of the other disclosed embodiment. For example, it is understood that any of the RNA molecules or compositions of the present invention may be utilized in any of the methods of the present invention.

As used herein, all headings are simply for organization and are not intended to limit the disclosure in any manner. The content of any individual section may be equally applicable to all sections.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization A Laboratory Course Manual” CSHL Press (1996); “Bacteriophage Methods and Protocols”, Volume 1: Isolation, Characterization, and Interactions, all of which are incorporated by reference. Other general references are provided throughout this document.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### EXPERIMENTAL DETAILS

### Example 1

### ADA2 Correction Analysis

Guide sequences comprising 17-22 nucleotides in the sequences of 17-22 contiguous nucleotides set forth in SEQ ID NOs: 1-12655 are screened for high on target activity. On target activity is determined by DNA capillary electrophoresis analysis.

According to DNA capillary electrophoresis analysis, guide sequences comprising 17-22 nucleotides in the sequences of 17-22 contiguous nucleotides set forth in SEQ ID NOs: 1-12655 are found to be suitable for correction of the ADA2 gene.

### Discussion

The guide sequences of the present invention are determined to be suitable for targeting the ADA2 gene.

### Example 2

### ADA2 Guide Activity Test

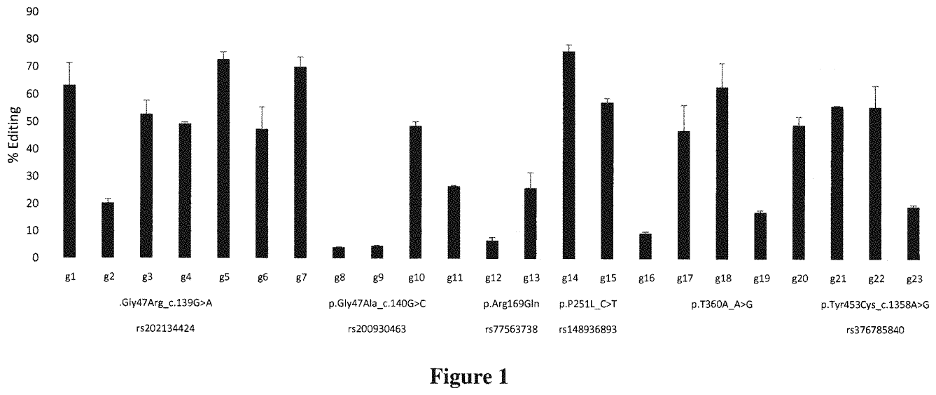

To choose the optimal guides for editing strategies in ADA2 indication, 23 different guides (Table 2), were screened for high on-target activity in HeLa cells, which are homozygous to the WT allele. The guides target mutations in different regions of ADA2, which relevant to the therapeutic editing strategies. Briefly, spCas9 coding plasmid (64 ng) was co-transfected with each of the guide DNA plasmids (20 ng) in 96 well plate format using jetOPTIMUS reagent (Polyplus). Cells were harvested 72 h post DNA transfection. Genomic DNA was extracted and used for capillary electrophoresis using primers amplify the endogenous genomic regions. The graph in FIG. 1 represents the average of % editing±STDV of 3 independent experiments. According to capillary electrophoreses analysis all guides show activity ranging between 5%-80%.

TABLE 2
Sequence of ADA2 guides

| Name | sgRNA | SEQ ID No. |
|---|---|---|
| DADA2-g1 | AAGAAAAGAUGAUGCGGCUG | 10879 |
| DADA2-g2 | AGAAAAGAUGAUGCGGCUGA | 11244 |
| DADA2-g3 | GAAAAGAUGAUGCGGCUGAG | 11263 |
| DADA2-g4 | AAAAGAUGAUGCGGCUGAGG | 11240 |
| DADA2-g5 | AGAUGAUGCGGCUGAGGGGG | 11245 |
| DADA2-g6 | GAUGCGGCUGAGGGGGCGGC | 11266 |
| DADA2-g7 | AGAAAAGAUGAUGCGGCUGG | 10756 |
| DADA2-g8 | GAAAAGAUGAUGCGGCUGGC | 11021 |
| DADA2-g9 | AAAAGAUGAUGCGGCUGGCG | 11000 |
| DADA2-g10 | AGAUGAUGCGGCUGGCGGGG | 11004 |
| DADA2-g11 | GAUGCGGCUGGCGGGGCGGC | 11023 |
| DADA2-g12 | UGCUGGAGGAUUAUCAGAAG | 8270 |
| DADA2-g13 | GCUGGAGGAUUAUCAGAAGC | 8255 |
| DADA2-g14 | GAUCAGAGCCAGGCUGCUGC | 6157 |
| DADA2-g15 | UGGGCUCACCAGCAGCAGCC | 6310 |
| DADA2-g16 | UUCUUCCACGCCGGAGAAGC | 2626 |
| DADA2-g17 | CGGAGAAGCAGGUGAGCCUG | 2601 |
| DADA2-g18 | GGAGAAGCAGGUGAGCCUGC | 2615 |
| DADA2-g19 | CGCAGGCUCACCUGCUUCUC | 2598 |
| DADA2-g20 | GCUCACCUGCUUCUCCGGCG | 2613 |
| DADA2-g21 | CUUGUCCUGUGAUUUCUAUG | 1164 |
| DADA2-g22 | GAAGACCUCAUAGAAAUCAC | 1166 |
| DADA2-g23 | GAAAUCACAGGACAAGCCUU | 1165 |

Guides characterized with activity lower than 10% according to the capillary electrophoresis were further tested by in vitro cleavage assay with Cas9 RNP and DNA template which contains the relevant mutation.

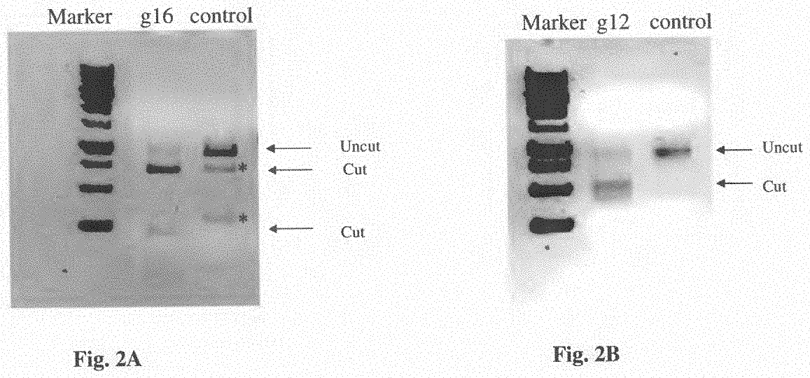

In short, in order to assemble RNP, 2 pmol gRNA were incubated with 2 pmol sp Cas9 for 10 min in 25° C. Next, the RNP was incubated for 1 hr in 37° C. with 150 ng dsDNA, PCR product substrate, that contains the relevant mutation. In order to determine activity, the samples were run on 1.7% agarose gel after proteinase K treatment. According to the results, g12 and g16 are active on the mutated template but not active on the WI allele, which suggests that sp Cas9 possess discrimination activity with g12 and g16 (See FIG. 2A and FIG. 2B.).

1. An RNA molecule comprising a guide sequence portion having 17-25 nucleotides comprising the sequence of 20-22 contiguous nucleotides set forth in any one of SEQ ID NOs: 1-12655.

2. The RNA molecule of claim 1, further comprising
a portion having a tracr mate sequence; and/or
a portion having a tracrRNA sequence which binds to a CRISPR nuclease, and/or one or more linker portions.

3. (canceled)

4. (canceled)

5. (canceled)

6. The RNA molecule of claim 1, wherein the RNA molecule is up to 300 nucleotides in length.

7. A composition comprising the RNA molecule of claim 1 and a CRISPR nuclease.

8. The composition of claim 7, further comprising a nucleic acid template for homology-directed repair, alteration, or replacement of at least a portion of a mutant ADA2 allele.

9. The composition of claim 7, further comprising a second RNA molecule comprising a guide sequence portion having 17-25 nucleotides comprising the sequence of 20-22 contiguous nucleotides set forth in any one of SEQ ID NOs: 1-12655, wherein the sequence of the guide sequence portion of the first RNA molecule is different from the sequence of the guide sequence portion of the second RNA molecule.

10. A method for correcting a mutant ADA2 allele in a cell, the method comprising delivering to the cell the composition of claim 7, wherein a complex of the CRISPR nuclease and the RNA molecule affects a double strand break in the mutant ADA2 allele, and/or wherein the CRISPR nuclease and the RNA molecule are delivered to the cells substantially at the same time or at different times.

11. (canceled)

12. (canceled)

13. (canceled)

14. The method of claim 8, wherein the nucleic acid template is delivered to the cells substantially at the same time or at different times as the CRISPR nuclease and RNA molecule or RNA molecules.

15. The method of claim 10, comprising obtaining the cell with a mutant adenosine deaminase 2 (ADA2) allele from a subject with a mutant ADA2 allele and which subject is (a) homozygous for the mutant ADA2 allele, or (b) heterozygous for the mutant ADA2 allele and a second, different mutant ADA allele.

16. The method of claim 15, comprising obtaining the cell from the subject by mobilization and/or by apheresis, or by bone marrow aspiration.

17. (canceled)

18. The method of claim 10, wherein the cell is prestimulated prior to introducing the composition to the cell.

19. The method of claim 15, further comprising culture expanding the cell to obtain cells.

20. The method of claim 19, wherein the cells are cultured with:
(a) one or more of: stem cell factor (SCF), IL-3, and GM-CSF; and/or
(b) at least one cytokine, wherein the at least one cytokine is preferably a recombinant human cytokine.

21. The method of claim 10, wherein delivering the composition comprises electroporation of the cell or cells.

22. A modified cell obtained by the method of claim 10.

23. (canceled)

24. The modified cell of claim 22, wherein the cell, or cells obtained from culture expanding the cell, are capable of:
(a) engraftment;
(b) giving rise to progeny cells;
(c) giving rise to progeny cells after engraftment;
(d) giving rise to progeny cells after an autologous engraftment; and/or
(e) giving rise to progeny cells for at least 12 months or at least 24 months after engraftment.

25. The modified cell of claim 22, wherein the modified cell is a hematopoietic stem cell and/or progenitor cell HSPC;
wherein the modified cell is a CD34+ hematopoietic stem cell; or
wherein the modified cell is a bone marrow cell or peripheral mononucleated cell (PMC).

26. (canceled)

27. A composition comprising the modified cell of claim 22 and a pharmaceutically acceptable carrier.

28. (canceled)

29. A method of treating a subject afflicted with Adenosine deaminase 2 (ADA2) deficiency, comprising administration of a therapeutically effective amount of the modified cells of claim 22.

30. (canceled)

31. (canceled)

32. (canceled)

33. A method of treating, ameliorating, or preventing Adenosine deaminase 2 (ADA2) deficiency, the method comprising delivering to a subject having or at risk of having Adenosine deaminase 2 (ADA2) deficiency the modified cell or cells of claim 24.

34. (canceled)

35. (canceled)

36. (canceled)

37. (canceled)
